(19) United States
(12) Patent Application Publication
(10) Pub. No.: US 2021/0339295 A1
(43) Pub. Date: Nov. 4, 2021

(54) Parallel conveyor belts, direct wireless charging systems utilizing artificial intelligence and machine learning

(71) Applicant: Keith Louis DeSanto, St Petersburg, FL (US)

(72) Inventor: Keith Louis DeSanto, St Petersburg, FL (US)

(21) Appl. No.: 17/368,966

(22) Filed: Jul. 7, 2021

Related U.S. Application Data
(63) Continuation of application No. 62/968,410, filed on Jan. 31, 2020.

(51) Int. Cl. B08B 6/00; B65G 47/92; B03C 3/34; G06N 20/00; B65G 43/08; G16H 50/20; G16H 50/70; B65G 45/22; B08B 7/00; B08B 7/04; H02J 50/30

(52) U.S. Cl. CPC B08B 6/00; B65G 47/92; B03C 3/34; G06N 20/00; B65G 43/08; G16H 50/20; B65G 2207/18; B65G 45/22; B08B 7/0035; B08B 7/0071; B08B 7/04; H02J 50/30; G16H 50/70

(57) ABSTRACT

This new application collects data from indoor and outdoor environments and with that data compiles databases, analyzes that data and finds relationships between pollutants, microbes, matter and diseases in humans, plants and animals. This new application is called the Artificial Intelligence Doctor. The application utilizes artificial intelligence, machine learning and parallel conveyor belts with imbedded microscope slides for the identification and analysis of microbes and matter. The application identifies microbes and matter using static electricity applied to microscope slides imbedded in conveyor belts using light microscopes, electron microscopes, polarized light microscopes, x ray machines, artificial intelligence and machine learning algorithms. The easy transfer conveyor belt system utilizes migration of microbes and microbes from drones and robots for easier identification.

This new application collects data from indoor and outdoor environments and with that data compiles databases, analyzes that data and finds relationships between pollutants, microbes, matter and diseases in humans, plants and animals. This new application is called the Artificial Intelligence Doctor. The application utilizes artificial intelligence, machine learning and parallel conveyor belts with imbedded microscope slides for the identification and analysis of microbes and matter. The application identifies microbes and matter using static electricity applied to microscope slides imbedded in conveyor belts using light microscopes, electron microscopes, polarized light microscopes, x-ray machines, artificial intelligence and machine learning algorithms. The easy transfer conveyor belt system utilizes migration of microbes and matter form drones and robots.

The application utilizes artificial intelligence and machine learning algorithms to evaluate, understand and forecast disease causing events for people, plants and animals in urban, rural and remote communities.

This new application utilizes artificial intelligence, machine learning, conveyor belts with imbedded microscope slides that run parallel to each other, pillows, fabric sheets, plastic sheets, rubber sheets and screens for the identification and analysis of pollutants, microbes and matter. The application collects, identifies, compiles information, data and databases, analyzes that data and predicts behaviors on airborne and surface pollutants, microbes and matter in singular and cluster form such as bacteria, molds, viruses, allergens, biological germs, different forms of dust and solids, dirt, grime, toxins, organic and inorganic material “data points”. The prior list of microbes and matter will be hereafter referred to as “microbes and matter”. The data collected is evaluated and the behaviors are learned by our new artificial intelligence and machine learning platform of algorithms. The data collected from this application also notes what was identified, the date it was identified, the time it was identified, geographically where it was identified, how it was identified (drone, robot, pillow, screen), what happened to the sample (was it saved or destroyed), what was stored (pictures, structure/building data) and where the data is stored such as which server, what part of the world the server is located and where the backup to the server is located. The artificial intelligence platform of algorithms also manages the entire application of software and hardware components.

This application utilizes slides imbedded in conveyor belts. This new application will have more than one (perpendicular to the ground) conveyor belts parallel to each, some sections are parallel to each other where others are end to end. The application uses microbe and matter migration to the conveyor belts from drones and robots. Once the plates or slides are close enough to each other, the microbes and matter migrate to and from each other using static electricity. Our prior USPTO application uses microscope slides that are placed on and retrieved from conveyor belts by drones, robots and mechanical arms. This new application transfers microbes and matter from drones and robots to the conveyor belt for identification. The microbes are transferred to the conveyor belt slides by positioning the drones and robots next to the slides on the conveyor belt. The transfer of microbes and matter can also be by a slight touch by the drone or robot to the slide on the conveyor belt. The application neutralizes the drones and robots and their pillows, fabric sheets, plastic sheets, rubber sheets and screens while the plates and slides on the conveyor belts are charged with static electricity. The conveyor belt can be used in a vertical direction as opposed to horizontal conveyor belts that operate horizontal to the ground. The slides and plates used in this application are imbedded in the conveyor belt. The name microscope slides and plates are used interchangeably in this application where both are imbedded in the conveyor belts. The plates are usually larger than microscope slides and are made of materials other than glass. Plates and microscope slides will be one and the same for purposes of this application. The imbedded slides are of different sizes and swivel when making the turn on the conveyor belt. For certain applications such as data collection from small residences, the conveyor belts are of the smaller size and the slides do not swivel and will be horizontal to the ground. Each conveyor belt may have different types and sizes of plates and microscope slides depending on the data being acquired. Longer and larger conveyor belts are required for large municipal buildings (connected or stand-alone buildings) and can have many different types of plates and slides. Large ships such as cruise ships or those utilized by the military will have large conveyor belts (some larger than 3 stories) with many different components and sizes and types of plates. These conveyor belts (municipal, cruise ship and military) will require the slides to swivel on the conveyor belt. There are more data points in municipal, cruise ship and military applications than those in small homes. An example of this would be the amount of different types of microbes and matter that are found on cruise ships that travel the world. The difference in the amount of people boarding and disembarking the cruise ship as opposed to a single residence where there is only one occupant. Also, the different types of microbes that cruise ship vacationers are exposed to are far more than those of a home owner that does not own a car and does most of their traveling to and from their homes by walking (another data point for our algorithms).

### OVERVIEW OF APPLICATION

The basic understanding of this new application is imbedded plates in the conveyor belts. The static electricity application has also been advanced to include pillows made from folded fabrics, plastics and rubbers to basic screens that attract microbes and matter. This new application imbeds those pillows and screens to the outside of the drones and robots. The drones and robots only need to either slightly touch or be in close vicinity to the slides in the conveyor belt to transfer microbes and matter obtained from indoor and outdoor environments.

This new application utilizes one or more combinations of different components that are listed below. One application component is the machine learning algorithms that learn about the behaviors of airborne and surface microbes and matter in the environment (indoor and outdoor) and predict behaviors and events that are caused by microbes and matter. Specifically, the machine learning algorithms learn about environmental occurrences that cause diseases in humans, plants and animals hereafter referred to as “threats”. Included in this list are specific artificial intelligence applications that manage identification processes, data bases of airborne and surface microbes and matter in singular form and in clusters and possible solutions to the threats identified. The data points gathered is the identity of such microbe and matter as type, any shape (cone, rod, oval, circle and string) size, length, width, height, color, weight, circumference and diameter or any other physical attribute that may occur in environments. Data points are also behaviors of people.

### Data Points and Human Behaviors

Our machine learning algorithms constantly learns even when drones and robots are not running scans. The AI algorithms scan the internet, purchased databases that are private and government databases to compile data on all things that represent human and animal behavior, events and statistical data of diseases. An example of this would be that of weekly hobby nights in the local high school of a small community. It is a known fact that materials used in hobby making are chemicals that cause disease in people, plants and animals. A data point that the artificial intelligence and machine learning algorithms, their associated software and hardware components (hereafter referred to as AI platforms) will calculate is the large amount of people in a small community that have been stricken with a disease that have attended that specific event such as a hobby night. The AI platforms uses already existing data as well as new data obtained from this new application. Already existing data will be defined as data in the public domain the AI platform compiles. New data will be data that is learned by the machine learning and artificial intelligence algorithms from this new application. An example of both is the following.

### Public Domain Data

Public domain data is where data bases of already existing data is uploaded to the AI platform of servers. This data can be purchased or is available from agencies such as the EPA, FDA, CDC or local and state governments. This data is statistical medical data such as people that have developed lung cancer in a small community where the data points out that they are nonsmokers, aren't exposed to employment hazards such as working in a chemical plant and their homes are not located in a hazard zone such as an industrial chemical facility. This data was compiled by the local medical and government authorities as well as local doctors.

### New “Learned” Data

New learned data is where the AI platforms and algorithms gather data from this new application. If a hobby night is advertised on the internet and the place of gathering is a local high school in a community, the AI platform makes a note to itself (machine learning). The AI platform will gather as much information from the internet such as social media postings of event, local and worldwide message boards, what was made and how was it made (what chemicals were used). Information regarding the high school can also be made. An example of this would be that historical information on West Haven High School can be obtained from Wikipedia by the AI platform. In September of 1963, the school was opened. During that time, the new AI platform will ascertain what building materials may have been used to construct the building such as asbestos. Then, the new application data is compiled. If the hobby night is still operational, the platform will identify what microbes and matter are in the space during hobby night (using imbedded plates on a conveyor belt) and what threat do they pose. All of this information is learned about environmental events, what microbes and matter (chemicals, pathogens and germs) are determined to be in the indoor structure as well as outside surrounding areas, what activities took place (behaviors), and what were the outcomes (did people develop lung cancer and when was it diagnosed).

Many different types of matter (pollutants) are found indoors. These include but are not limited to pesticides, gases, fragrance-related compounds, polychlorinated biphenyls (PCBs), phthalates, combustion byproducts, carbon monoxide, benzene, formaldehyde, asbestos among other undesired compounds. Pollutants in indoor environments come from many different sources, including combustion sources such as furnaces, gas stoves, fireplaces, and cigarettes; building materials and furnishings such as treated wood, paints, furniture, carpet, and fabrics; consumer goods such as electronics and toys; cleaning products, pesticides, and other products used for maintenance of the home or facility; as well as other products used for hobbies, science projects, arts and crafts projects, and other activities.

There are other environmental occurrences that are broadcast in the public domain such as epidemics and pandemics when its too late. There are also signs that our AI platforms take note of while some human behaviors go unnoticed. The spread of a disease (virus) by animals, spread by people (fecal to oral transmission) or bugs (a bite from a mosquito). Norovirus, West Nile virus and rabies are example of this. Our application will alert restaurants that fecal matter and urine were found on dining room tables which eventually made their way to the kitchen (particular human behaviors). Occurrences may be that of mutations, variants, new synthetic chemicals (or combinations or clusters thereof) or a significant health incident in the community (possible epidemics, pandemics) that will need to be learned by the algorithms and how to handle them. This new occurrence will trigger a red alert and government contacts, medical contacts, military and law enforcement contacts will be notified immediately through the artificial intelligence platform by pre-arraigned contact preferences.

### The Hardware in the Application

Hardware components consist of the following and may be increased in number, size and combinations of each for different applications. Direct Wireless Charging Systems “DWCS”, lasers, cantilevers, high definition lenses, polarized light microscopes, light microscopes, electron microscopes, x ray machines, folded mirror lenses, conveyor belts imbedded with many different sizes and materials of microscope slides, pillows made from fabric, plastic or rubber, mesh for holding the pillows, screens, drones and robots. Included in the hardware list are also ancillary components listed in my prior USPTO applications such as servers and transmission equipment.

### How the Application Works

Microbes and matter are attracted to fabric, plastic, rubber and screens on drones and robots using static electricity. Then those microbes and matter are transferred to a conveyor belt for identification using high definition lenses over the conveyor belt, on the side of the conveyor belt, on top of the conveyor belt or under the conveyor belt even if the conveyor belt is vertical. The high definition lenses use the same components as a desktop microscope. Sometimes the microscope condenser will not be needed where other times, light, polarized and electron microscopes with condensers will be used in the same conveyor belt. Other times, x-ray machines will be needed to detect smaller matter. Our new folded mirror lenses may also be used in certain applications. These decisions are made by the artificial intelligence platform. This new application also utilizes different ways to have microbes and matter become attracted to the drones, robots and plates on the conveyor belt for identification. Basically, the slide on the conveyor belt is rubbed or touched by a fabric on the drone or robot thereby transferring some microbes and or matter to the slide.

There are 4 types of microbe and matter catchers applications;
1. Pillows
2. Sheets
3. Screens
4. DWCS ramps

Microbe and matter attraction using static electricity, fabric, plastic, rubber or screen application with and without pillow formation. For purposes of this application, the fabrics, plastics, or rubbers will be referred to as a “repository or repositories”. The repositories can be sheets, folded sheets or checkered with both. The definition of triboelectricity is an electrical charge developed by rubbing objects together, such as amber on cat fur. This application is based on this principal.

### Pillows

Pillows are defined for purposes of this application as a square, circle or any shape after two or more fabrics, plastics or rubbers are folded around each other to form a pillow or repository. The repository can be enclosed in a screen, mesh or string or wire to maintain the folds. Two or more sheets of different material (not two of the same fabric or plastic touch each other) must be rubbed together to hold microbes and matter in the folds. When the pillow is touched slightly, depressed, then released (creates a small amount of suction which this action alone pulls microbes and matter into the pillow) after being depressed or vibrated, static electricity is created. This static electricity charge attracts microbes and matter. The pillow works by being affixed to the drone or robot by a simple Velcro strip, 3 m dual lock reclosable fasteners or a basket for the pillow that can be affixed by the Velcro or 3 m dual lock for an easy replacement. Mechanical arms managed by the artificial intelligence platform manages the replacement of the pillow.

Magnetic fields also attract microbes and matter. The screen of mesh can be magnetized and demagnetized by an electromagnetic machine as in current flowing in a conductor will produce a magnetic field.

The pillow can also be used like a rag where a simple wiping of a surface will obtain matter and microbes. No static electricity is needed.

### Transfer of Microbes and Matter from Drones and Robots to the Conveyor Belt

In order for microbes and matter to be transferred from repositories, we must negate the static electricity while creating static electricity on the plates of the conveyor belt. Static electricity can be negated in several ways. The simplest way is to wave a metal object (metal hanger) over the area. Each of our conveyor belts are equipped with an application to negate the static electricity on the repositories. Before the drone or robot is ready to transfer the microbes and matter, the conveyor belt has a metal pole right where the drones and robots position themselves to negate the charge. On some applications, metal plates (in lieu of glass plates) imbedded in the conveyor belts will de-charge and pull the microbes and matter to the metal plates by using parallel conveyor belts where the metal plates are static electricity charged and the original conveyor belt plates were neutralized of static electricity. If the application requires a reverse transfer of microbes and matter, then the metal plates can be de-charged where the glass plates can also pull the microbes and matter from the metal plates. This application uses two conveyor belts running in parallel to each other. This creates an extra step where the climate may be non-conducive to microbe and matter collection due to sunshine, heat and high humidity to name a few challenges.

Static electricity is created for the application by using a wool pad that slightly touches the plate (metal or glass) on the conveyor belt.

If some of the microbes or matter were obtained by the wiping method will not transfer using any of the prior methods, the AI platform also uses the wipe method to the plates. This application also utilizes two conveyor belts parallel to each other with a very small space between them. The dual conveyor belt application touches the repositories and screens slightly to transfer the microbes or matter to the plates on the conveyor belt.

### Migration of Microbes and Matter

The transfer of microbes and matter to the conveyor belt uses the migration method. This is used by placing the drone and robot repository close to the slide on the conveyor belt where the electrons and or protons migrate from the repository to the imbedded plate on the conveyor belt. Static electricity is the imbalance of positive and negative charges. This is the loss or addition of protons and electrons from a pillow made up of fabric, plastic or rubber. No touching of the repository or screen to the plate needs to take place. The plates on the conveyor belts also process a static electricity charge so when other microbes and matter are placed in parallel and in close vicinity to the slides on the conveyor belt, the microbes and matter are pulled to the slide. Another application to remove static electricity from the drones and robots is to use a metal wand affixed to the end of the conveyor belt (on the frame) before the drone and robot get close to the slides on the conveyor belt.

### Sheets

Sheets (not folded) of fabric (wool) hard plastic (acrylic), soft thin flexible plastic films (sheets of plastic wrap such as saran wrap) or rubber repositories can also be used to attract microbes and matter to the drones and robots. The attachment of either of them to the drone or robot makes for an easy attraction of microbes and matter by simple vibration to collect airborne matter. Surface microbes and matter can be collected by slightly touching them to a surface while in motion. The transfer of atoms from and to the sheets creates the static electricity. Prop wash (defined as a current of air created by the action of a propeller or rotor) from drones pulls in microbes and matter while the vibration of the robots with knobby tires also creates the static electricity.

### Screens

Screens can be made of plastic, metal or any other material that will hold a static electric charge. The screens are also affixed on the outside of the drone or robot for microbe and matter attraction. The screens work in two ways having the screens magnetically charged or by creating a static electricity charge on them also attracts microbes and matter to the screens. The screens can be made of plastic, metal, wood or a combination of each.

### DWCS—Direct Wireless Charging Systems that Creates Static Electricity for Microbe and Matter Attraction

Our new DWCS application creates static electricity through the electrical current traveling through the charging mats that are placed on the ground, walls and ceilings of indoor structures as well as outdoors. For purposes of this application, mats and runners and pads will mean one and the same. A DWCS has a power source (listed in my prior USPTO application) that sends current through the connected mats that charge drones and robots while they are in motion without using wires. For purposes of this application, electricity is used as the power source. The drones and robots traveling over the interconnected mats can charge their batteries while in motion. This application also creates four distinct new inventions.
1. Creating a static electric charge on top of the mats for microbe and matter attraction.
2. Creating airborne microbes and matter that were attracted to the surface of the mat(s) by flying drones and driving robots over the mats.
3. Creating static electricity ramps where the open ramps (like that of a declined loading ramp for trucks) are positioned at intervals of the mats for drones and robots to scoop up microbes and matter from the bottom of the ramps.
4. Creating static electricity and transferring static electricity to the drones and robots by touching pads and pillows affixed to the drones and robots with the mats and or squares.

### Creating a Static Electric Charge on Top of the Mats

The current that travels through the connected mats creates static electricity. It is well known that electric current traveling through wires can create a magnetic field. Our special DWCS identification application is constructed where static electricity is not cancelled by metal poles on the sides of the mats but left to create a field that is beneficial to microbe and matter attraction without being hazardous. Static electricity exists in our hair, on our clothes and on everyday surfaces where at worst, a non-life-threatening shock will occur. The field will attract microbes and matter in a more efficient way since the current through the mat is dual and can be regulated. One current is to charge the drones and robots while the other current specially creates static electricity. The way this part of the application works is anywhere along the top of the mat matter and microbes accumulate. A simple touch of the any part of a repository or screen attached to the drone or robot should result in obtaining some type of microbe or matter. As with our prior USPTO applications, many runs by drones and robots should gather enough data for the accurate identity of microbe or matter. Some areas will have very little microbes and or matter (some have none) that are hazardous to people, plants and animals.

### Creating Airborne Microbes and Matter That Were Attracted to the Surface of the Mats by Flying Drones and Driving Robots Over the Mats

Creating clusters of microbes and matter by prop wash from drones and the vibration of robots in motion for identification purposes. This application learns about clusters of microbes and matter, what microbe is eating other microbes for nourishment, what makes up the cluster (the matter may be organic and inorganic together) and what else has become airborne and what is clinging to it. The machine learning algorithms learn about why some microbes tend to attach to certain matter and why some microbes and matter tend to repel each other.

### Creating Static Electricity Squares

Creating static electricity through drive and drop squares that make the process of gathering microbes and matter easier by simply setting the whole drone down in to the square or driving the robot into the square where the repository or screen (the screen is able to collect more microbes and matter than repositories in certain environments) cause a hard rub to transfer the microbes and matter. Once the drone or robot enter the square, the static electric field is reversed in the square while the static electric field remains on the repository or screen for easy microbe and matter transfer.

### Creating Static Electricity and Transferring Static Electricity to the Drones and Robots by Touching Repositories Affixed to the Drones and Robots with the Mats and or Squares

This application works in different ways. It utilizes a slight touch of fabric.

Obtaining surface microbe and matter for identification utilizes fabric formed like a pillow for touching a surface. The drones or robots touch the DWCS mat or pad on the ground that also carries electrical current and maintains its own sections of static electricity (also managed by the AI platform). The drones and robots will touch with their fabric certain sections of the DWCS mats or indiscriminate spaces on the mats all determined by the AI platform.

Another way is by prop wash from the drones. The air flow enables microbes and matter to become caught in the fabric affixed to the outside of the drone. The fabric on both drones and robots can be made of wool, polyester, cotton, canvas, cashmere, chenille, chiffon, silk, crepe, damask, georgette, gingham, jersey, lace, linen, merino, modal, muslin, organza, satin, spandex, suede, taffeta, toile, tweed, twill, velvet, viscose, or a sheet of plastic such as an plastic apron that when the two ends are rubbed together, the rubbing creates static electricity. Fabric and plastic or a combination of two or more materials may also be used for the creation of static electricity. The mat itself from the DWCS will also maintain microbes and matter on its surface and by the drones and robots traveling over it will cause those microbes and matter to become airborne. On some occasions, the fabric will not need to make contact the DWCS pad in order to collect those microbes and matter. This application utilizes screens affixed to the outside of the drones and robots. The screens may be attached to fabric or stand-alone without any fabric attached to them.

The materials used in this application are listed below

The fabric and sheets of plastic must be interlapped. Different fabric like wool and plastic when rubbed together create static electricity.

Plastic sheets can be that of plastic aprons or saran wrap (cling wrap, stretch wrap)

Rubber sheets (styrene)

Plastic squares such as vinyl, polycarbonate, acrylic, carbon fiber, corrugated plastic or industrial plastics such as acrylonitrile, polyvinyl chloride “pvc” and polypropylene.

Fabric sheets such as wool, polyester, cotton, canvas, cashmere, chenille, chiffon, silk, crepe, damask, georgette, gingham, jersey, lace, linen, merino, modal, muslin, organza, satin, spandex, suede, taffeta, toile, tweed, twill, velvet and viscose.

The above-named materials form the plastic sheets folded around the fabric into pillows

The above-named materials form the fabric sheets folded around the plastic into pillows

Some pillows or plastic sheets will be imbedded (checkered) with fabric and some fabric sheets will be imbedded (checkered) with pieces of plastic or rubber.

The plastic can be a hard solid or a flexible square or in a sheet like fabric. Some applications may have more than two fabrics and or plastic folded or stacked together.

Fabric may also be wrapped around plastic aprons folded inside the fabric pillow. Or, a plastic apron material (thin sheets of plastic) may be folded around fabric. Either way, static electricity is created by the slightest movement between the sheets. In some occasions, fabric is checkered on top of a plastic apron or vice versa. There are many ways to create static electricity.

Fabric or sheets of plastic can be charged with static electricity by several ways.

Microbes and matter get caught in fabric attached to the drone and robots. The drones and robots then transfer the microbes and matter from the fabric to the imbedded microscope slides in the conveyor belts. The transfer is done by a simple touch of the fabric to the slides. The drone and robot can set themselves right next (or underneath or top of) to or at the ends of the conveyor belt where the turning conveyor belt touches the fabric on the drone or robot. The touching of the fabric with a slight degree of movement from the prior touch may transfer some different, some of the same or no microbes or matter at all. With many touches, the more data acquired and the more accurate the data results.

### Suction from Fabric Pillow Application

When the fabric of the pillow is compressed and then released it creates a suction where the microbes and or matter are pulled in towards the fabric. The drone and robot at times will put pressure on the fabric. That suction application is also managed by the artificial intelligence and machine learning platform. An example would be if the sample of matter or microbe in the atmosphere is in high humidity, prop wash will be needed for attraction. The pressure would be more to compensate for limited static electricity. Another example would be if the sample of matter or microbe on a surface is in an atmosphere is in very low humidity, the pressure would be less by the robot or drone. Drones would have to shut down when not using the prop wash application and a pillow would be used and compressed by the weight of the drone.

In some applications, the fabric moves from the bottom to the side or the top of both the drone and robot by an electronic device (like that of an elevator) managed by the artificial intelligence platform. The drones and robots can transfer the microbes and matter “pillow” either by placing themselves by the side or underneath the conveyor belt. The drones and robots can also maintain a mesh box that houses the fabric pillow. This application is two-fold where both the pillow and the mesh can both attract microbes and matter. Obtaining airborne microbe and matter for identification utilizes fabric in mesh wired boxes that can be disposable. The mesh can be of various materials from wood, to plastics to metals. Meshes can be molded into squares, rectangles and circles.

### Worn or Dirty Fabric

The fabric can be replaced, washed or blown, or simply heated while on the drone and robot. The replacement is done by mechanical arms or by the docking station. The docking station utilizes an application akin to a razor in a razor cartridge. A simple docking of the drone and robot will replace the pillow.

### Preserving the Integrity of Bacteria, Viruses and Biological Germs Using Enclosures

In order to not taint the current sample of microbe and matter, the application may need to enclose the conveyor belt to keep the data accurate. The system uses erected shed or tent enclosures that may be used by the application for outdoor microbe and matter data collection. The indoor tent or shed uses two different types of enclosures, one with four sections where the conveyor belt and DWCS are sectioned in an airtight enclosure where the entrance and exit from the enclosure have a separate entrance and exit and opens and closes with the entrance and exit of robots and drones. Another type of enclosure can be a singular structure. The indoor application may not need the enclosures. The AI platform determines and manages whether or not an enclosure is required by what type of microbe or matter identity is needed, what type of environment (i.e. indoor, outdoor, high winds, humidity, toxic) the microbe or matter is in (hostile, battlefield) and geographically where the application is being placed.

The tent or shed enclosure is made of fabric such as nylon, rubber, plastic, canvas, polyester poly vinyl chloride, plastic or vinyl shed, aluminum, steel, wood or any other material that a tent or shed can be made of. The atmosphere of the tent or shed may need to be altered and the pumping of oxygen into tent or shed may be needed if a more accurate identification of the solid is needed. The enclosure can be airtight by just using plastic around the outside or inside of the structure. Other times, the air inside the structure may need to be heated or cooled by a heater or air conditioner. Lights at times can be used to heat the structure or, the structure can be completely black inside. A dehumidifier may also be needed. The walls may be magnetized or have an electro static charge applied to attract other microbes and pollutants to the walls as to not interfere with the identity process by the spinning of the conveyor belt. A vacuum may also put inside the enclosure with or without pure filtered oxygen being also pumped into the enclosure. The AI application manages the process by utilizing machine learning algorithms that have learned from prior application data. Cameras are mounted on the inside of the structure for monitoring purposes. The monitoring can be from short or far distances. Miniature robots with mechanical arms stand by for assistance if needed. Robots can be normal sized or nanotechnology or a combination of both.

### Cleaning Plates and Conveyor Belts with Lasers, Brushes and Vacuums

Our new application uses lasers to clean the plates and the conveyor belt in lieu of using a liquid to clean them. Our lasers can kill on contact bacteria, viruses and molds. The high heat from the lasers also does away with some pollutants that are on the slides and conveyor belts.

### The Cleaning Application

Cleaning specification with lasers, brushes and vacuums.

Or a brush with a vacuum removes most of the pollutants after the laser kills them.

Our new application uses lasers to clean the plates on the conveyor belt and sometimes when required, the entire area in lieu of using a liquid to clean them. Our lasers can kill on contact bacteria, viruses, molds. The high heat from the lasers also does away with pollutants that remain on the slides. The cleaning application can also use a brush with a vacuum that removes most of the pollutants after the laser kills them. The brush and vacuum can also be used independently of the lasers. The AI platform makes those decisions as to what is needed to clean the platform before and if new data is needed or acquired.

The slide can turn 360 degrees. Tiny turning machines are at one or each end of the slide. A vertical conveyor belt swivels and can swivel the plates 360 degrees without any help due to gravity and hinges at one end of the slide. The swivel can be placed at a certain degree for a cleaning brush (see below) to clean the slide. The machines may also be powered by springs that maintain tension from the turning of the conveyor belt located at each end of the slide. The slide can also be made of metal or sections of the slide can be metal where metal-based pollutants can be obtained for evaluation and identity. The sections of metals on the slides can be magnetized for obtaining some pollutants.

### Cleaning of the Slides and the Conveyor Belt

The cleaning brush can be mounted to the conveyor belt or is attached to a mechanical arm independent of the conveyor belt. The cleaning brush or numerous brushes are used to clean both the slides and all components of the entire conveyor belt.

The cleaning brush may be cleaned of microbes and matter by the 5 following methods:

The bristles of the brush can be cleaned by heat,

Placed in a vacuum tube and cleaned,

Cleaned by using static electricity by a cloth placed on the bristles

or by a stationary fabric pad

or by the mechanical arm utilizing another brush called a catch

or by a laser that obliterates the microbe or matter.

An expensive application replaces the cleaning brush or brushes at certain intervals of time and or rotations of the conveyor belts.

A more expensive conveyor belt cleaning application can have the entire conveyor belt being sprayed with a stream of pressured air. The application utilizes an enclosure.

A brush may be made of different materials to create static electricity by the bending and touching of the bristles with each other. The different bristle materials can be that of plastic, rubber, fabric coated or many other materials where when rubbed together create a static electricity charge to attract microbes and matter from slides and the conveyor itself.

Brushes last from 1 week to 12 months before needing replacement depending upon the amount type of microbe and matter buildup. The artificial intelligence platform with machine learning algorithms will determine if and when the cleaning brushes need cleaning or replacement. This is done through cameras and sensors located in and around the conveyor belt.

The most basic type of cleaning would be a sprayed (misted liquid) non-toxic detergent or chemical (Rhamnolipid) by force (pressure) to clean the entire belt, plates and conveyor belt frame.

### Miniature Conveyor Belt

Our application also utilizes a conveyor belt that is small in size where it can be placed by a drone and or robot at locations to collect data. The small sized conveyor belt application with all the above components can be cleaned by forced air or a vacuum. The application can also use miniature components discussed in this application or some or all of the components can use nano-technology.

The screen on the drones and robots can also be of miniature size where the microbes and matter get caught in a screen where high definition lenses identifies it through algorithms and high def pictures from folded mirrored lenses, micron microscope lenses or electron microscope lenses. Nanotechnology sized mechanical arms work with this application.

The artificial intelligence and machine learning platform “AI application” will also forecast some disease-causing events in humans, animals and plants. An example of this would be an event involving a venus fly trap nursery that was exposed to swimming pool that was 200 feet from the nursery. The chorine that was added to the filter system of the pool was found to have killed the venus fly traps. The application not only noted high levels of chorine on the surface of the nursery, but the chorine became airborne due to the climate of high humidity.

1. An application using direct wireless charging systems, lasers, cantilevers, high definition lenses, polarized light microscopes, light microscopes, electron microscopes, x-ray machines, conveyor belts imbedded with many different sizes and materials of microscope slides and plates, static electricity, pillows, fabric sheets, plastic sheets, rubber sheets, screens, cleaning brushes, docking stations, tent and shed enclosures, vacuums, humidifiers, dehumidifiers, air compressors, heat lights, artificial intelligence and machine learning algorithms, drones and robots to identify microbes, matter and pollutants that cause disease in people, plants and animals.

2. An application to migrate airborne and surface microbes, matter and pollutants from indoor and outdoor environments gathered from drones and robots to a conveyor belt using static electricity, artificial intelligence and machine learning algorithms.

3. An application where a direct wireless charging system has two functions, one for electrical current that charges the batteries of drones and robots while the other creates static electricity for the attraction of microbes and matter.

4. An application in claims 1 through 3 where the microbe and matter migration application combined with the direct wireless charging system application and machine learning and artificial intelligence algorithms that compile databases of environmental occurrences, databases of behaviors of human and animals can predict the spread and cause of diseases.

5. An application in claims 1 through 4 where the algorithms are used to learn what types of microbes and matter are in the atmosphere, what types of microbes and matter are on surfaces, what specific data compiled from environments can cause disease in humans, plants and animals, which data compiled from environments caused disease in humans, plants and animals and what types of events in an environment can cause disease in humans, plants and animals.

6. An application in claims 1 through 5 where the algorithms learn from microbes and matter from indoor and outdoor environments that have various characteristics and shapes that include cones, rods, ovals, circles, strings, size, length, width, height, color, weight, circumference and diameter.

7. An application in claims 1 through 6 where the machine learning and artificial intelligence algorithms compile internal application data and data from public available databases such as scanning the internet, scanning private databases, federal databases, CDC, FDA and EPA databases and local and state governmental databases to learn about causes of disease in people, plants and animals.

8. An application in claims 1 through 7 where the entire system of platforms working together can predict outbreaks of disease and disease causing events.

9. An application in claim 1 where there are more than two conveyor belts where the length of the conveyor belts run between the floors of an indoor structure and can be over 25 feet in length and the conveyor belts overlap each other by being exactly parallel to each other or some sections overlap each other or the conveyor belts are end to end.

10. An application where high definition lenses, electron microscopes, light microscopes, polarized light microscopes and x ray machines are placed on the top of, the side of or beneath a conveyor belt perpendicular or horizontal to the ground or floor.

11. An application in claims 1 and 2 where the direct wireless charging system creates static electricity for the attraction of microbes and matter and also neutralizes static electricity when required.

12. An application in claims 1 and 2 where the direct wireless charging system creates static electricity zones to attract microbes and also neutralizes static electricity from those zones.

13. An application in claims 1 and 2 where static electricity is created and used to attract microbes and matter to pillows, screens, sheets of fabric, sheets of plastic and sheets of rubber that are affixed to the outside of drones and robots.

14. An application in claims 1 and 2 where drones and robots using pillows, fabric, plastic and screens attract microbes and matter using static electricity from indoor and outdoor environments and transfers those microbes and matter to a conveyor belt through migration by placing the drones and robots in close vicinity to the conveyor belts.

15. An application in claim 14 to migrate microbes and matter from drones and robots to a conveyor belt by negating the static electricity on the drones and robots and by creating static electricity on the plates and slides imbedded in a conveyor belt.

16. An application in claims 14 and 15 where the slides and plates on conveyor belts, come between 1/64 of an inch to 2 and 1/16 of an inch of drones and robots having pillows, sheets, fabric sheets, plastic sheets, rubber sheets and screens for microbe and matter migration.

17. An application in claims 1 through 16 where an airtight enclosure or sections of connected enclosures such as a tent or a shed encloses the entire conveyor belt operation to maintain a clean environment of accurate testing.

18. An application in claims 1 and 2 where the plates or slides imbedded in the conveyor belt, the entire conveyor belt or the sheets of fabric, sheets of plastic, sheets of rubber or screens on the drones and robots can be cleaned of microbes and matter using one or more components of the following such as a mist or spray of non-toxic detergents and carriers mixed with rhamnolipid, lasers, brushes with and without static electricity, heat, vacuums, compressed air, or heat lights.

19. An application in claim 1 where a folded mirror lens is used.

20. An application in claims 1 through 19 where the system can be nano-technology sized, normal sized or a combination of both.
